(19) United States
(12) Patent Application Publication
(10) Pub. No.: US 2026/0060687 A1
(43) Pub. Date: Mar. 5, 2026

(54) METHOD OF TREATING HEART FAILURE IN A PATIENT

(71) Applicant: ABIOMED, INC., DANVERS, MA (US)

(72) Inventors: SATHYA DEV UNUDURTHI, DANVERS, MA (US); ALFRED KYROLLOS, DANVERS, MA (US); LARA REYELT, DANVERS, MA (US); NAVIN KAPUR, DANVERS, MA (US); JERALD CURRAN, DANVERS, MA (US)

(73) Assignees: ABIOMED, INC., DANVERS, MA (US); TUFTS MEDICAL CENTER, INC., BOSTON, MA (US)

(21) Appl. No.: 19/310,435

(22) Filed: Aug. 26, 2025

Related U.S. Application Data
(60) Provisional application No. 63/687,684, filed on Aug. 27, 2024.

(51) Int. Cl. A61B 17/12

(52) U.S. Cl. CPC A61B 17/1204; A61B 17/12031; A61B 17/12109; A61B 17/12136

(57) ABSTRACT

A method of treating heart failure in a patient includes delivering an occlusion device through the vasculature into the Superior Vena Cava (“SVC”) of the patient. The occlusion device is activated within the SVC such that blood flow from the SVC is effectively continuously occluded for a period of about 6 hours. After the 6-hour period of time, the occlusion device within the SVC is deactivated. The occlusion device is removed from the vasculature of the patient.

### CROSS REFERENCE TO RELATED APPLICATIONS

This application claims the benefit of priority under 35 U.S. C. § 119 to prior filed U.S. Provisional Ser. No. 63/687,684 , filed Aug. 27, 2024 (Attorney Docket No.: 266489.000009 (ABD0416USPSP1)), the entire contents of which is hereby incorporated by reference in its entirety as if set forth in full herein.

### BACKGROUND

The present disclosure generally relates devices and methods for improving cardiac function in patients suffering from heart failure.

Heart failure, including Acute Decompensated Heart Failure (“ADHF”), is a major cause of global mortality. Heart failure often results in multiple long-term hospital admissions, especially in the later phases of the disease. Absent heart transplantation, the long-term prognosis for such patients is not very good, and pharmaceutical approaches are palliative only. Consequently, there are few effective treatments to slow or reverse the progression of this disease.

Heart failure can result from any of multiple initiating events. Heart failure may occur because of ischemic heart disease, hypertension, valvular heart disease, infection, inherited cardiomyopathy, pulmonary hypertension, or under conditions of metabolic stress including pregnancy. Heart failure also may occur without a clear cause, also known as idiopathic cardiomyopathy. The term heart failure encompasses left ventricular, right ventricular, or biventricular failure.

While the heart can often initially respond successfully to the increased workload that results from high blood pressure or loss of contractile tissue, over time this stress induces compensatory cardiomyocyte hypertrophy and remodeling of the ventricular wall. Over the next several months after the initial cardiac injury, the damaged portion of the heart typically will begin to remodel as the heart struggles to continue to pump blood with reduced muscle mass or less contractility. This in turn often leads to overworking of the myocardium, such that the cardiac muscle in the compromised region becomes progressively thinner, enlarged and further overloaded. Simultaneously, the ejection fraction of the damaged ventricle drops, leading to lower cardiac output and higher average pressures and volumes in the chamber throughout the cardiac cycle, the hallmarks of heart failure. Not surprisingly, once a patient's heart enters this progressively self-perpetuating downward spiral, the patient's quality of life is severely affected and the risk of morbidity increases. Depending upon a number of factors, including the patient's prior physical condition, age, sex and lifestyle, the patient may experience one or several hospital admissions, at considerable cost to the patient and social healthcare systems, until the patient dies either of cardiac arrest or any of a number of co-morbidities including stroke, kidney failure, liver failure, or pulmonary hypertension.

Pulmonary hypertension (PH) is also a major cause of morbidity and mortality worldwide. While heart failure is a common cause of pulmonary hypertension, as mentioned above, pulmonary hypertension may also be caused by primary lung disease. Today, pharmacologic treatments may reduce pulmonary artery systolic pressure (PASP) and improve symptoms and ultimately survival for patients with pulmonary hypertension. However, there are drawbacks to pharmacologic treatments such as costs and side effects.

In view of the foregoing drawbacks of the previously known systems and methods for regulating venous return to address heart failure, it would be desirable to provide systems and methods for treating acute and chronic heart failure that reduce the risk of exacerbating co-morbidities associated with the disease.

It further would be desirable to provide systems and methods for treating acute and chronic heart failure that arrest or reverse cardiac remodeling and are practical for chronic and/or ambulatory use.

It still further would be desirable to provide systems and methods for treating heart failure that permit patients suffering from this disease to have improved quality of life, reducing the need for hospital admissions and the length of hospital stays, and the associated burden on societal healthcare networks.

It also would be desirable to provide systems and methods that permit treatment of pulmonary hypertension and cardiorenal syndrome.

### SUMMARY

In view of the drawbacks of the previously known systems and methods for treating heart failure, it would be desirable to provide systems and methods for treating acute and/or chronic heart failure that can arrest, and more preferably, reverse cardiac remodeling that result in the cascade of effects associated with this disease.

It further would be desirable to provide systems and methods for arresting or reversing cardiac remodeling in patients suffering from heart failure that are practical for ambulatory and/or chronic use.

It still further would be desirable to provide systems and methods for treating heart failure that reduce the risk of exacerbating co-morbidities associated with the disease, such as venous congestion resulting in renal and hepatic complications.

It also would be desirable to provide systems and methods for treating heart failure that permit patients suffering from this disease to have improved quality of life, while reducing the need for hospital re-admissions and the associated burden on societal healthcare networks.

It further would be desirable to provide systems and methods for treating pulmonary hypertension that permit patients suffering from this disease to have improved quality of life. In addition, it would be desirable to provide systems and methods for treating heart attacks, acute heart failure, chronic heart failure, heart failure with preserved ejection fraction, right heart failure, constrictive and restrictive cardiomyopathies, and cardio-renal syndromes (Types 1-5).

A method of treating heart failure in a patient includes delivering an occlusion device through the vasculature into the Superior Vena Cava (“SVC”) of the patient. The occlusion device is activated within the SVC such that blood flow from the SVC is effectively continuously occluded for a period of about 6 hours. After the 6-hour period of time, the occlusion device within the SVC is deactivated. The occlusion device is removed from the vasculature of the patient.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1A is a frontal, partially broken-away view of the major arteries and veins of the heart.

FIG. 1B illustrates the vena cava including major veins associated with the vena cava.

FIG. 2 is a view of an introducer sheath entering an SVC, and a flow occluding element within the SVC.

FIG. 3 is an illustration of the vasculature with a pressure sensor disposed in the circle of Willis.

FIG. 4 is chart illustrating cerebral blood pressure, which closely co-relates with intracranial pressure, as compared to aortic MAP.

FIG. 5 is a picture of a brain of a patient showing that SVC occlusion for an extended time does not cause cerebral edema or hemorrhage as seen in gross morphological analysis as compared to a control brain.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the term “microcatheter” is a catheter having a diameter that is small in comparison to catheters in cardiovascular applications, i.e. 8 French or less. As used herein, a measurement of 1 French is equivalent to 1/3 mm, or 3 mm is equivalent to 1 French.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Mean arterial pressure (“MAP”) is defined as the average pressure in a patient's arteries during one cardiac cycle. To calculate MAP, double the diastolic blood pressure (“DBP”) and add the sum to the systolic blood pressure (“SBP”). Then divide by 3. For example, if a patient's blood pressure is 83 mm Hg/50 mm Hg, the MAP would be 61 mm Hg. Here are the steps for this calculation:

MAP=SBP+2(DBP)/3

MAP=83+2(50)3

MAP=83+100/3

MAP=183/3

MAP=61 mm Hg

Another example of a way to calculate the MAP is to first calculate the pulse pressure (subtract the DBP from the SBP) and divide that by 3, then add the DBP:

MAP=⅓(SBP−DBP)+DBP

MAP=⅓(83−50)+50

MAP=⅓(33)+50

MAP=11+50

MAP=61 mm Hg

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

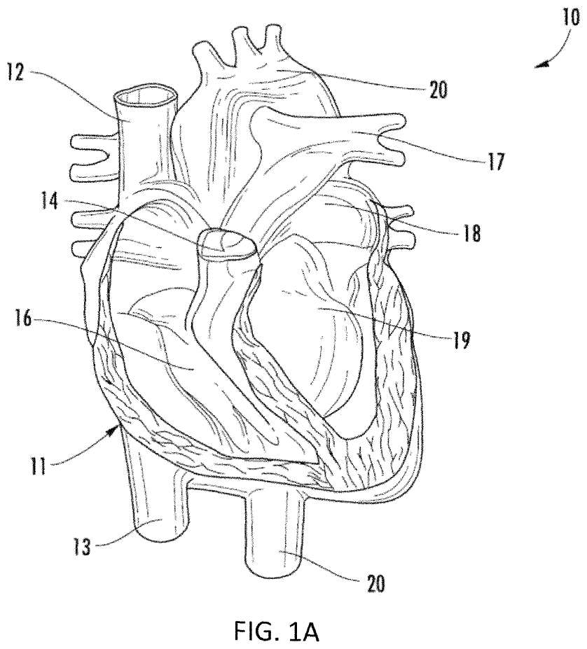

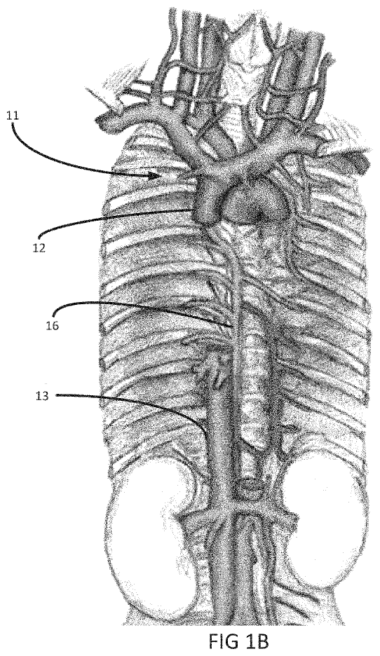

Referring to FIGS. 1A and 1B, the human anatomy in which the present disclosure is designed for placement and operation is described as context for the system and methods of the present disclosure.

More particularly, referring to FIG. 1A, deoxygenated blood returns to the heart 10 through vena cava 11, which comprises superior vena cava 12 and inferior vena cava 13 coupled to right atrium 14 of the heart. Blood moves from the right atrium 14 through the tricuspid valve to the right ventricle 16, where it is pumped via the pulmonary artery 17 to the lungs. Oxygenated blood returns from the lungs to the left atrium 18 via the pulmonary vein. The oxygenated blood then enters the left ventricle 19, which pumps the blood through aorta 20 to the rest of the body.

As shown in FIG. 1B, the superior vena cava (“SVC”) 12 is positioned at the top of the vena cava 11, while the inferior vena cava (“IVC”) 13 is located at the bottom of the vena cava. FIG. 1B also shows the azygos vein and some of the major veins connecting to the vena cava. As noted herein, occlusion of the IVC 13 may pose risks of venous congestion, and in particular, potential blockage or enlargement of the hepatic veins and/or suprarenal vein that may worsen, rather than improve, the patient's cardiovascular condition and overall health. It should be noted that approximately 70% of the blood entering the vena cava comes from the IVC and the remaining 30% comes from the SVC. Thus, even if the SVC is fully occluded, about 70% of the normal blood flow will still enter the left ventricle. This permits the heart to be partially unloaded during the treatment in accordance with the present disclosure.

In accordance with one aspect of the present disclosure, continuous occlusion of the SVC poses fewer potential adverse risks than continuous occlusion of the IVC. Moreover, animal testing reveals that controlling the return of venous blood to the right ventricle by fully occluding the SVC beneficially lowers right ventricular end-diastolic pressure (“RVEDP”), right ventricular end-diastolic volume (“RVEDV”), left ventricular end-diastolic pressure (“LVEDP”) and left ventricular end-diastolic volume (“LVEDV”) without adversely reducing left ventricular systolic pressure (“LVSP”). See, for example, Appendices A and B, the disclosure of which is hereby fully incorporated by reference.

An example of the method can show that continuous occlusion of the SVC will reduce the risk of worsening congestion of the kidneys, which is a major cause of ‘cardio-renal’ syndrome, as compared to IVC occlusion. Cardio-renal syndrome is impaired renal function due to volume overload and neurohormonal activation in patients with heart failure. Volume overload may occur where the weakened heart cannot pump as much blood, which leads to less blood flow through the kidneys. With less blood flow through the kidneys, less blood is filtered by the kidneys and less water is released via urination causing excess volume to be retained in the body. With the excess volume, the heart pumps with increasingly less efficiency and the patient ultimately spirals toward death as the body becomes progressively more congested.

IVC occlusion generally reduces the blood flow through the kidneys as the occluded IVC increases pressure in the renal vein, thereby reducing the kidneys'ability to filter out fluid. IVC occlusion further causes blood to back-up and otherwise prevents deoxygenated blood from returning to the heart. As a result, renal function may too be reduced, worsening congestion. However, SVC occlusion ultimately increases flow to the kidneys thereby improving renal function. Specifically, by reducing flow into the right atrium via SVC occlusion, volume within the left ventricle is ultimately reduced, permitting the muscle fibers to stretch within a normal range, naturally increasing contractility and allowing the heart to drive more fluid to the kidneys. The kidneys may then extract water, which may be removed from the body through urination. It is further understood that during SVC occlusion, a negative pressure sink is created in the right atrium caused by an abrupt reduction in right atrial pressure and volume. As a result, flow from the renal vein may be accelerated thereby enhancing renal decongestion and promoting blood flow across the kidney, increasing urine output. Accordingly, continuous SVC occlusion may benefit patients with heart failure and/or cardiorenal syndrome by reducing cardiac and pulmonary pressures and promoting decongestion.

In addition, implantation in the SVC permits a supra-diaphragmatic device implant that could not be used in the IVC without cardiac penetration and crossing the right atrium. Further, implantation of the occluder in the SVC avoids the need for groin access as required by IVC implantation, which would limit mobility making an ambulatory device impractical for short term or long-term use. In addition, minor changes in IVC occlusion (time or degree) may cause more dramatic shifts in preload reduction and hence total cardiac output/systemic blood pressure whereas the systems and methods of the present disclosure are expected to permit finely tuned decrease in venous return (i.e., preload reduction).

Continuous occlusion of the SVC (i.e., cardio-pulmonary unloading) over a period of time (e.g., minutes, hours, days, weeks, or months) will beneficially permit a patient's heart to discontinue or recover from remodeling of the myocardium. Animal testing indicates that the method and system of the present examples enables the myocardium to transition from pressure-stroke volume curve indicative of heart failure towards a pressure-stroke volume curve more closely resembling that of a healthy heart.

In general, the system and methods of the present disclosure may be used to treat any disease to improve cardiac function by arresting or reversing myocardial remodeling, and particularly those conditions in which a patient suffers from heart failure. Such conditions include, but are not limited to, e.g., systolic heart failure, diastolic (non-systolic) heart failure, acute decompensated heart failure (“ADHF”), chronic heart failure, acute heart failure and pulmonary hypertension, heart attacks, heart failure with preserved ejection fraction, right heart failure, constrictive and restrictive cardiomyopathies, and cardio-renal syndromes (Types 1-5). The system and methods of the present disclosure also may be used as a prophylactic to mitigate the aftermath of acute right or left ventricle myocardial infarction, pulmonary hypertension, RV failure, post-cardiotomy shock, or post-orthotopic heart transplantation (“OHTx”) rejection, or otherwise may be used for cardiorenal applications and/or to treat renal dysfunction, hepatic dysfunction, or lymphatic congestion. Also, the system and methods of the present disclosure may reduce hospital stays caused by various ailments described herein, including at least acute exacerbation.

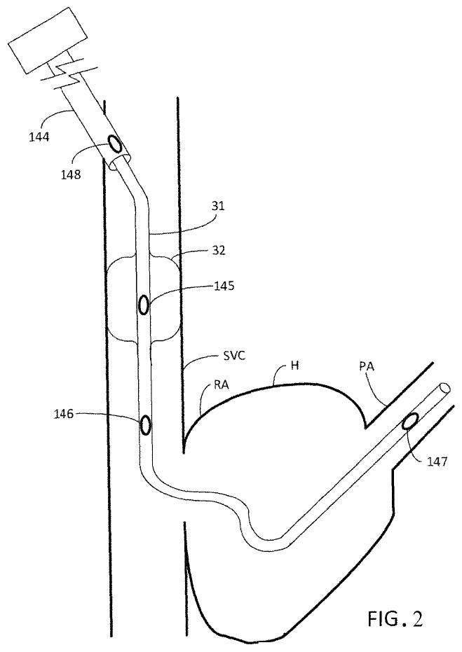

Referring now to FIG. 2 which illustrates an introducer sheath 144 entering the SVC, and a flow occluding element 32 being disposed within the SVC. In accordance with one aspect of the present invention, an occluding system can be a preCARDIA™ system, which is a catheter-based system that includes a superior vena cava balloon that can intermittently or continuously occlude the SVC. The preCARDIA™ system is commercially available from Abiomed, a Johnson & Johnson company. The occluding system includes a catheter 31 that extends into the SVC. The system can include sensors 145, 146, 147, 148 to measure pressure. Sensor 148 can measure the Jugular Vein Pressure (JVP) while sensor 145 can measure pressure within the flow occluding element 32 (i.e., balloon pressure). Sensor 146 can measure the SVC pressure or the right atrium pressure while sensor 147 can measure the pulmonary artery pressure. Flow occluding element 32 can be connected to a control system that is external to the patient and as is known in the art (incorporated by reference in its'entirety is U.S. Pat. No. 10,842,974). The control system (not illustrated) can include, among other things, a power source, a processor, an inflation and deflation source, and a data transfer module to selectively inflate and deflate the flow occluding element 32.

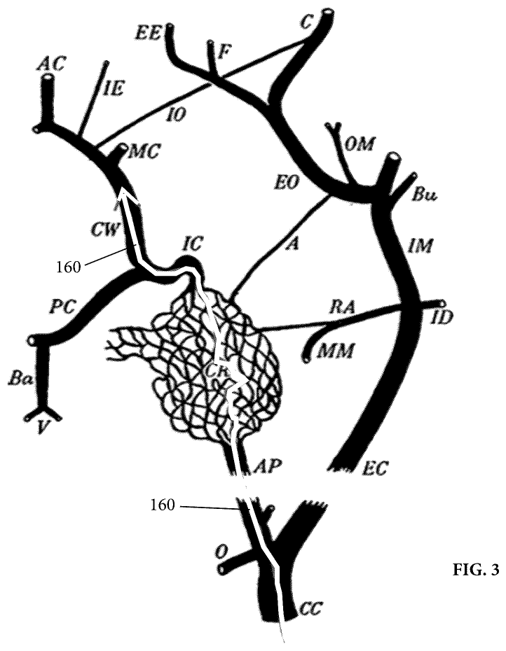

Referring now to FIG. 3 a schematic illustration of the vasculature is shown. A pressure sensor is disposed on a distal end of a wire 160 in the circle of Willis (“CW”). To place wire 160 in the CW, a diagnostic catheter can be passed along a guide wire up the common carotid artery (“CC”) into the ascending pharyngeal artery (“AP”) and then advanced into the Rete mirabile. A hydrophilic coated wire can be transversed past the rete mirabile into the internal carotid artery (“IC”) and into the circle of Willis CW. Then a low-profile catheter can be floated over the wire. Once past the Rete Mirabile with the low-profile catheter, a pressure wire 160 can be exchanged for continuous blood pressure measurement in the CW.

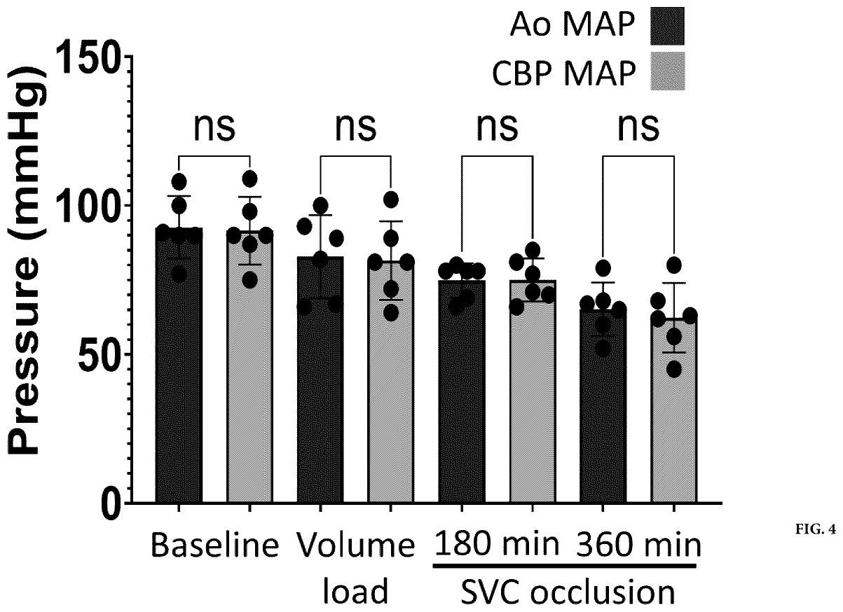

Referring now to FIG. 4, a bar chart showing cerebral blood pressure (“CBP”) MAP, which closely co-relates with intracranial pressure, as compared to aortic (“Ao”) MAP is illustrated. The chart shows on the x-axis, the Ao MAP and the CBP MAP at baseline (before occlusion of the SVP), at volume load, at 180 minutes after occlusion of the SVP starts, and at 360 minutes after occlusion of the SVP starts. The pressure in mmHg is shown on the y-axis. In the chart, a comparison of the Ao MAP to the CBP MAP is shown at 180 minutes after occlusion of the SVP and at 360 minutes after occlusion of the SVP by the balloon catheter. As can be seen, the MAP measured at about 3 hours after occlusion starts is approximately 80% of the baseline MAP. The MAP measured at about 6 hours is approximately 70% of the baseline MAP.

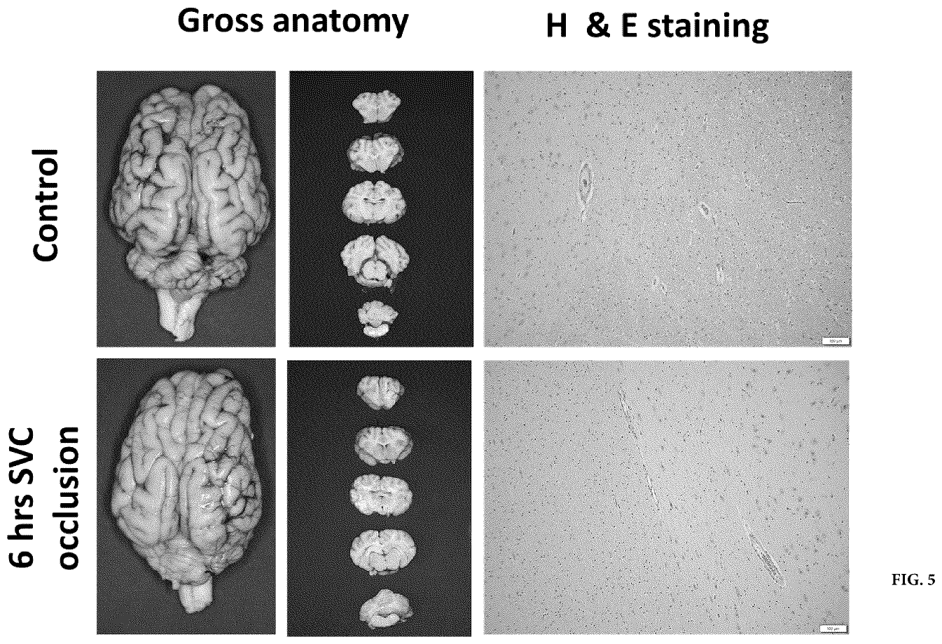

Referring now to FIG. 5, images of two brains are presented. The top image is a picture of a control brain, that is one that has not undergone SVC occlusion. The bottom image is a picture of a brain of a patient who had undergone 6 continuous hours of SVC occlusion is illustrated. To the left of the whole brain images is the same brain sectioned to show interior tissue. The image to the farther left is a section of the brain tissue that had been H & E (“hematoxylin and eosin”) stained. Visual comparison of the three control images to the patient images illustrates little to no visual signs of trauma to the patient's brain. The picture shows that SVC occlusion for an extended time up to about 6 hours does not cause cerebral edema or hemorrhage as seen in gross morphological analysis as compared to a control brain.

In accordance with the disclosure a method of treating heart failure in a patient is disclosed. The method includes delivering an occlusion device through the vasculature into the SVC of the patient. The occlusion device is activated within the SVC such that blood flow from the SVC is effectively continuously occluded for a period of about 6 hours. Thereafter, the occlusion device is deactivated within the SVC. The occlusion device is then removed from the vasculature of the patient.

The cerebral blood pressure of the patient is measured during the 6-hour occlusion period. The cerebral blood pressure of the patient can be measured at approximately one-hour intervals during the 6-hour occlusion period. Alternatively, the cerebral blood pressure of the patient can be measured at more or less frequent intervals, such as, for example, at 30-minute intervals or even continuously during the 6-hour occlusion period. The cerebral blood pressure is maintained at acceptable levels during the 6-hour occlusion period. While blood flow is effectively occluded in the SVC, blood flow in the IVC is maintained uninterrupted during the 6-hour occlusion period of the SVC to maintain cardiac output.

The MAP of the patient can be measured during the 6-hour occlusion period in the SVC. The measured MAP can be compared to a baseline MAP. The baseline MAP is determined before the activating the occlusion device. The measured MAP can be compared to the baseline MAP approximately every 60 minutes. The measured MAP can also be compared continuously to the baseline MAP. Of course, MAP can be measured at more or less frequent intervals, such as, for example, at 30-minute intervals during the 6-hour occlusion period. The measured MAP should be maintained between about 50 and about 150 mmHg during the 6-hour occlusion period.

The MAP can be measured using a minimally invasive closed system. The cerebral blood pressure can be measured, for example, proximate to the circle of Willis. In one example, the cerebral blood pressure is measured in the circle of Willis. The minimally invasive closed system can be a wire having pressure sensor disposed on a distal end of the wire.

The occlusion device can be a mechanical occlusion device. In one example, the mechanical occlusion device is a balloon catheter. The activating step includes inflating the balloon catheter. The balloon is movable between a first radially compressed state and a second radially expanded state. In the radially expanded state the SVC is effectively occluded. The balloon in the first radially compressed state may have an outer diameter of about 3.0 mm (9 French). In other embodiments, the balloon in the first radially compressed state may have an outer diameter of about 10 French. As will be appreciated, the balloon may have any suitable outer diameter in the first radially compressed state. The balloon in the second radially expanded state has an outer diameter between about 30 mm and about 36 mm and can further have a volume of about 20mL. As will be appreciated, the balloon may have any suitable outer diameter in the second radially expanded state. As will be appreciated, any suitable media may be used to inflate the balloon. In some instances, a contrast saline media may be used. In such instances, up to 20mL of contrast saline media may be used. However, as will be appreciated, any suitable volume of media may be used to inflate the balloon.

The patients can be animals or humans. In one example, the patients are Sus scrofa domesticus, which is commonly known as pigs.

The intracranial pressure of the patient can be measured during the 6-hour occlusion period. In addition, the systolic, diastolic, and mean aortic pressures of the patient can be measured during the 6-hour occlusion period. The systolic, diastolic, and mean femoral pressures of the patient can also be measured during the 6-hour occlusion period. A jugular vein pressure of the patient can be measured during the 6-hour occlusion period. A pulmonary artery pressure of the patient can be measured during the 6-hour occlusion period. A thermal dilution cardiac output of the patient can be measured during the 6-hour occlusion period. A renal vein pressure of the patient can be measured during the 6-hour occlusion period. Blood gases from at least one of a carotid artery, cranial internal jugular vein, arterial or a venous blood of the patient can be measured during the 6-hour occlusion period. A urine output of the patient can be measured during the 6-hour occlusion period. A glial fibrillary acidic protein (“GFAP”) of the patient during the 6-hour occlusion period. A ubiquitin carboxy-terminal hydrolase L1 (UCH-L1) of the patient can be measured during the 6-hour occlusion period.

The measuring of all of these parameters can occur at approximately thirty-minute intervals during the 6-hour occlusion period. Alternatively, the measuring occurs at approximately one-hour intervals during the 6-hour occlusion period.

A trans-valvular heart pump can be inserted into the heart of the patient during the 6-hour occlusion period to further assist the heart. The trans-valvular heart pump can be, for example, an Impella® heart pump, currently available from Abiomed, a Johnson & Johnson company. The trans-valvular heart pump is inserted into the left ventricle of the heart.

A method of reducing biventricular pressures while maintaining cardiac output of a heart of a patient, can include the steps of: delivering an occlusion device into the SVC of the patient; continuously occluding the SVC with the occlusion device such that blood flow from the SVC is effectively prevented from entering the heart for a period greater than 2.5 hours; deactivating the occlusion device within the SVC; and removing the occlusion device from the patient.

The SVC can be continuously occluded from greater than 2.5 hours to about 6 hours. The SVC can be continuously occluded from greater than or equal to 3.0 hours. In one example, the SVC is continuously occluded for about 6 hours.

The mean arterial pressure (“MAP”) of the patient can be measured. The baseline MAP is determined before the activating the occlusion device. Thereafter, the MAP is measured at intervals during the continuous occlusion step. The measured MAP is compared to the baseline MAP. The measured MAP is compared to the baseline MAP at least one of continuously or approximately every 60 minutes. The measured MAP is between about 50 and about 150 mmHg during the continuous occlusion step. The measured MAP at about 3 hours after initiating the occlusion is approximately 80% of the baseline MAP. The measured MAP at about 6 hours after initiating the occlusion is approximately 70% of the baseline MAP.

A trans-valvular heart pump is inserted into a left ventricle of the heart of the patient during the occluding step.

The continuous occluding of the SVC does not cause at least one of cerebral edema, ocular edema, cerebral hemorrhage, or neurological damage.

The disclosed technology can be further understood according to the following clauses:
1. A method of treating heart failure in a patient, the method comprising:
   delivering an occlusion device through the vasculature into the Superior Vena Cava (“SVC”) of the patient;
   activating the occlusion device within the SVC such that blood flow from the SVC is effectively continuously occluded for a period of about 6 hours; and deactivating the occlusion device within the SVC.
2. The method of clause 1, further comprising: measuring a cerebral blood pressure of the patient during the 6-hour occlusion period.
3. The method of clause 2, wherein the cerebral blood pressure of the patient is measured at approximately one-hour intervals during the 6-hour occlusion period.
4. The method of clauses 2-3, wherein the cerebral blood pressure of the patient is maintained above 60 mmHg during the 6-hour occlusion period.
5. The method of clauses 1-4, further comprising:
   permitting blood flow in the Inferior Vena Cava (“IVC”) during the 6-hour occlusion period of the SVC to maintain cardiac output.
6. The method of clauses 1-5, further comprising:
   measuring the mean arterial pressure (“MAP”) of the patient during the 6-hour occlusion period in the SVC.
7. The method of clause 6, wherein the measured MAP is compared to baseline MAP, wherein the baseline MAP is determined before the activating the occlusion device.
8. The method of clauses 1-7, wherein the measured MAP is compared to the baseline MAP approximately every 60 minutes.
9. The method of clauses 1-7, wherein the measured MAP is compared continuously to the baseline MAP.
10. The method of clauses 1-9, wherein the measured MAP is between about 50 and about mmHg during the 6-hour occlusion period.
11. The method of clauses 1-10, wherein the MAP is measured using a minimally invasive closed system.
12. The method of clauses 1-11, wherein the cerebral blood pressure is measured proximate to the circle of Willis.
13. The method of clauses 1-12, wherein the cerebral blood pressure is measured in the circle of Willis.
14. The method of clauses 1-13, wherein the minimally invasive closed system is a wire having pressure sensor disposed on a distal end of the wire.
15. The method of clauses 1-14, wherein the occlusion device is a mechanical occlusion device.
16. The method of clauses 1-15, wherein the mechanical occlusion device is a balloon catheter.
17. The method of clauses 1-16, where in the activating step includes inflating the balloon catheter.
18. The method of clauses 1-17, wherein the balloon is movable between a first radially compressed state and a second radially expanded state.
19. The method of clause 18, wherein the balloon in the first radially compressed state has an outer diameter of about 3.0 mm (9 French).
20. The method of clauses 18-19, wherein the balloon in the second radially expanded state has at least one of an outer diameter of about 30 mm to about 36 mm or a volume of about 20mL.
21. The method of clauses 1-20, wherein the patients are animals.
22. The method of clause 21, wherein the patients are Sus scrofa domesticus.
23. The method of clauses 1-22, further comprising:
   measuring an intracranial pressure of the patient during the 6-hour occlusion period.
24. The method of clauses 1-23, further comprising:
   measuring a systolic, diastolic, and mean aortic pressures of the patient during the 6-hour occlusion period.
25. The method of clauses 1-24, further comprising:
   measuring a systolic, diastolic, and mean femoral pressures of the patient during the 6-hour occlusion period.
26. The method of clauses 1-25, further comprising:
   measuring a jugular vein pressure of the patient during the 6-hour occlusion period.
27. The method of clauses 1-26, further comprising:
   measuring a pulmonary artery pressure of the patient during the 6-hour occlusion period.
28. The method of clauses 1-27, further comprising:
   measuring a thermal dilution cardiac output of the patient during the 6-hour occlusion period.
29. The method of clauses 1-28, further comprising:
   measuring a renal vein pressure of the patient during the 6-hour occlusion period.
30. The method of clauses 1-29, further comprising:
   measuring at least blood gases from at least one of a carotid artery, cranial internal jugular vein, arterial or a venous blood of the patient during the 6-hour occlusion period.
31. The method of clauses 1-30, further comprising:
   measuring a urine output of the patient during the 6-hour occlusion period.
32. The method of clauses 1-31, further comprising:
   measuring a glial fibrillary acidic protein (GFAP) of the patient during the 6-hour occlusion period.
33. The method of clauses 1-32, further comprising:
   measuring a ubiquitin carboxy-terminal hydrolase L1 (UCH-L1) of the patient during the 6-hour occlusion period.
34. The method of clauses 23-33, wherein the measuring occurs at approximately thirty-minute intervals during the 6-hour occlusion period.
35. The method of clauses 23-33, wherein the measuring occurs at approximately one-hour intervals during the 6-hour occlusion period.
36. The method of clause 1, further comprising the step of inserting a trans-valvular heart pump into the heart of the patient during the 6-hour occlusion period.
37. The method of clause 36, wherein the trans-valvular heart pump is inserted into the left ventricle of the heart.
38. The method of clause 1, wherein the heart failure is Acute Decompensated Heart Failure (ADHF).
39. A method of reducing biventricular pressures while maintaining cardiac output of a heart of a patient, comprising the steps of:
   delivering an occlusion device into the Superior Vena Cava (“SVC”) of the patient;
   continuously occluding the SVC with the occlusion device such that blood flow from the SVC is effectively prevented from entering the heart for a period greater than 2.5 hours; and
   deactivating the occlusion device within the SVC.
40. The method of clause 39, wherein the SVC is continuously occluded from greater than 2.5 hours to about 6 hours.
41. The method of clause 39, wherein the SVC is continuously occluded from greater than or equal to 3.0 hours.
42. The method of clause 41, wherein the SVC is continuously occluded for about 6 hours.
43. The method of clauses 39-42, further comprising:
   measuring the mean arterial pressure (“MAP”) of the patient, comprising:
      measuring a baseline MAP, wherein the baseline MAP is determined before the activating the occlusion device; and measuring the MAP at intervals during the continuous occlusion step.
44. The method of clause 43, wherein the measured MAP is compared to the baseline MAP.
45. The method of clause 44, wherein the measured MAP is compared to the baseline MAP at least one of continuously or approximately every 60 minutes.
46. The method of clauses 43-45, wherein the measured MAP is between about 50 and about 150 mmHg during the continuous occlusion step.
47. The method of clauses 43-46, wherein the MAP measured at about 3 hours is approximately 80% of the baseline MAP.
47. The method of clauses 43-47, wherein the MAP measured at about 6 hours is approximately 70% of the baseline MAP.
49. The method of clause 39, further comprising the step of inserting a trans-valvular heart pump into a left ventricle of the heart of the patient during the occluding step.
50. The method of clauses 39-49 wherein the continuous occluding step does not cause at least one of cerebral edema, ocular edema, cerebral hemorrhage, or neurological damage.
51. The method of clauses 1-50, further comprising the step of removing the occlusion device from the patient.

The descriptions contained herein are examples of embodiments of the disclosure and are not intended in any way to limit the scope of the disclosure. As described herein, the disclosure contemplates many variations and modifications of a method of reducing biventricular pressures while maintaining cardiac output of a heart of a patient. Modifications and variations apparent to those having skill in the pertinent art according to the teachings of this disclosure are intended to be within the scope of the claims which follow.

What is claimed is:

1. A method of treating heart failure in a patient, the method comprising:
delivering an occlusion device through the vasculature into the Superior Vena Cava (“SVC”) of the patient;
activating the occlusion device within the SVC such that blood flow from the SVC is effectively continuously occluded for a period of about 6 hours; and
deactivating the occlusion device within the SVC.

2. The method of claim 1, further comprising:
measuring a cerebral blood pressure of the patient during the 6-hour occlusion period.

3. The method of claim 2, wherein the cerebral blood pressure of the patient is measured at approximately one-hour intervals during the 6-hour occlusion period.

4. The method of claim 3, wherein the cerebral blood pressure of the patient is maintained above 60 mmHg during the 6-hour occlusion period.

5. The method of claim 1, further comprising:
permitting blood flow in the Inferior Vena Cava (“IVC”) during the 6-hour occlusion period of the SVC to maintain cardiac output.

6. The method of claim 1, further comprising:
measuring the mean arterial pressure (“MAP”) of the patient during the 6-hour occlusion period in the SVC.

7. The method of claim 6, wherein the measured MAP is compared to baseline MAP, wherein the baseline MAP is determined before the activating the occlusion device.

8. The method of claim 7, wherein the measured MAP is compared to the baseline MAP approximately every 60 minutes.

9. The method of claim 7, wherein the measured MAP is compared continuously to the baseline MAP.

10. The method of claim 9, wherein the measured MAP is between about 50 and about 150 mmHg during the 6-hour occlusion period.

11. The method of claim 10, wherein the MAP is measured using a minimally invasive closed system.

12. The method of claim 2, wherein the cerebral blood pressure is measured proximate to the circle of Willis.

13. The method of claim 2, wherein the cerebral blood pressure is measured in the circle of Willis.

14. The method of claim 11, wherein the minimally invasive closed system is a wire having pressure sensor disposed on a distal end of the wire.

15. The method of claims 1, wherein the occlusion device is a mechanical occlusion device.

16. The method of claim 15, wherein the mechanical occlusion device is a balloon catheter.

17. The method of claim 16, where in the activating step includes inflating the balloon catheter.

18. The method of claim 17, wherein the balloon is movable between a first radially compressed state and a second radially expanded state.

19. The method of claim 18, wherein the balloon in the first radially compressed state has an outer diameter of about 3.0mm (9 French).

20. The method of claim 19, wherein the balloon in the second radially expanded state has at least one of an outer diameter of about 30 mm to about 36 mm or a volume of about 20 mL.
